(19) United States
(12) Patent Application Publication
(10) Pub. No.: US 2022/0062361 A1
(43) Pub. Date: Mar. 3, 2022

(54) Herbal Supplement Composition and Method for Improved Digestion of Food

(71) Applicant: Suresh MAHAWAR, Fremont, CA (US)

(72) Inventor: Suresh MAHAWAR, Fremont, CA (US)

(21) Appl. No.: 17/008,726

(22) Filed: Sep. 1, 2020

(51) Int. Cl. A61K 36/235; A61K 35/02; A61K 9/20; A61K 9/48; A61K 9/14; A61K 9/00; A23L 33/105; A23L 33/00

(52) U.S. Cl. CPC A61K 36/235; A61K 35/02; A61K 9/20; A23L 33/30; A61K 9/14; A61K 9/0053; A23L 33/105; A61K 9/48

(57) ABSTRACT

The present invention relates to a herbal supplement composition for improved digestion of food in a subject in need having range of ingredient concentrations comprising asafoetida between 0.1 gm and 5 gm; Foeniculum vulgare seeds powder between 5 mg and 15 gm; Trachyspermum ammi seeds powder between 5 mg and 15 gm; and shilajit powder between 5 mg and 300 mg. Furthermore, the present invention relates to a method of improving digestion of food in a subject in need comprising orally administering a composition containing asafetida, Foeniculum vulgare seeds powder, Trachyspermum ammi seeds powder and shilajit powder.

### TECHNICAL FIELD

The present invention generally relates to a composition for improving the process of digestion of food. More specifically, the present invention discloses an herbal supplement based composition and method to enhance the food digestion process in human beings.

### BACKGROUND OF THE INVENTION

From olden days, our ancestors have been using various traditional herbal medicines to cure various illnesses in human beings. The advantage of using herbal medicines is that they involve minimal industrial processing and they are natural based. They are derived from plants so they do not affect adversely to the health of human beings, if taken in optimum quantity. Our ancestors have been using these herbs for digestion for thousands of years. These natural herbs do not harm us if they are consumed in optimum amounts. The herbs based medicines involve aromatic properties which help in curing headaches and other diseases. There are many herbal medicines which our ancestors have been using since ancient period for treating stomach disorders. Like Turmeric, it is used to reduce effects of diseases like dyspepsia, ulcerative colitis etc. Thus, plants based products play pivotal role in the health care sectors. The Indian system of herbal based medicines, also known as Ayurveda, is mainly focused on the development of methods, process of formulations of drugs from plants to cure various ailments in human beings.

Indigestion and flatulence are very common ailment that happens to most of us. The popular causes of flatulence are overeating, smoking, eating incompatible products same time and poor meal timing etc. Flatulence is not any disease but is considered to be part of the normal digestion process. If it happens excessively and frequently, then it could be an indication of an internal disorder such as hormonal imbalance, bowel obstruction, and excessive acid production by the stomach. Our ancestors have been using Trachyspermum ammi, or asafetida to help such stomach problems.

If we look into the Indian system of medicine, there are various compositions which include Trachyspermum ammi, Foeniculum vulgare seeds and asafetida to ease gassiness or indigestion. A brief introduction about these spices is given below:

Trachyspermum ammi helps in reducing flatulence or digestion problems. It contains compound thymol which helps in which helps in secreting gastric juices and maintaining better digestion. It contains bitter and pungent taste and strong aroma.

Asafetida has anti-flatulent, antiseptic, anti-inflammatory properties. It is very beneficial for easing gassiness and indigestion. Asafetida also helps in keeping blood pressure under control.

Foeniculum vulgare, is a medicinal herb which has distinctive taste with full of flavors. It is green and white, with feathery leaves and yellow flowers. The seeds of the Foeniculum vulgare plant have a mild, licorice-like flavor which is more potent due to their powerful essential oils. Foeniculum vulgare seeds contain an anti-spasmodic, anti-bacterial, anti-microbial and anti-inflammatory property that helps to stimulate the production of gas in the stomach and gives quick relief from stomach problems like bloating and gas.

The combination of above mentioned spices helps in curing various ailments. All of three ingredients (Asafetida, Foeniculum vulgare, & Trachyspermum ammi) help reduce blood Pressure & asthma. Foeniculum vulgare & Asafetida helps maintaining healthy skin. Foeniculum vulgare reduces Inflammation and Cancer due to its antioxidant property. Asafetida relieves Headache, Ear Pain, & Abdominal Pain (Intestinal or Menstrual Cramps). According to ancient Ayurveda, asafetida balances Vayu, Pitta, & Cough. Trachyspermum ammi helps fight fungal and bacterial infection.

One more important element that can be used with above composition to reduce bloating and gas in the stomach. Shilajit contains folic acid and various minerals, so it offers countless health benefits. It can function as an antioxidant to improve your body's immunity and memory. It can be used as an anti-inflammatory, an energy booster, and a water pill to remove excess fluid from your body. Moreover, it can be used as a drug that helps in the digestion and absorption of food in the gastrointestinal tract and is also useful for the treatment of nausea, vomiting, and digestive disorders.

Apart from these spices, there is a juice called apple cider vinegar which helps to improve digestion. It produces the enzyme called pepsin which helps to break down the protein which is produced from protein based heavy meals. This process helps to improve digestion. Drinking apple cider vinegar on an empty stomach increases health benefits and maximizes ability to process food.

Thus there is a need to combine all above mentioned ingredients in one which can be orally administered to provide ease from digestive problems.

Indian patent application IN201811011711A discloses a novel herbal formulation/composition for the management of diabetes mellitus. The composition relates to Amalaki (Emblica officinalis); Jambu (Syzygium jambolanum); Methi (Trigonella foenum); Upkunchikaa (Nigella sativa); Ajamoda (Trachyspermum mum ammi); Tejapatra (Cinnamomum zeylanicum), Pistacia species; Sunthi (Zingiber officinale); Haritaki (Terminalia chebula); Vibhitaka (Terminalia bellirica); Shilajit; and Maricha (Piper nigrum) and process of preparing the same.

U.S. Pat. No. 6,080,401A discloses curative action of drugs, including herbal remedies, allopathic remedies, and periodontal remedies, is enhanced and accelerated by administering such drugs in combination or association with probiotics, especially those of genus Lactococcus, Lactobacillus, Pediococcus, Streptococcus, Propionibacterium, Brevibacterium, Penicillium, and Saccharomyces.

Another WIPO Patent WO2018027081 A1 discloses nutraceutical composition including a neuroprotecting composition, an anti-inflammatory composition, a cardiovascular protecting composition, an antioxidant composition, and a gelling component in a sufficient amount to provide a cohesive gelled product.

Indian Patent Application IN201811008460A discloses an ayurvedic herbo-mineral formulation for treating type-2 diabetes by clearing excess of lipids from peripheral human body cells. The formulation contains 105 ingredients. A method for the preparation of the formulation according to any of the preceding claims, said method includes the following steps: (a) Mixing of all the components; (b) Drying the mixture obtained from step (a) to prepare the final powder form of said formulation.

Another US Patent Application discloses herbal formulation for treatment and management of metabolic disorders and related complications. The disclosed herbal formulation includes herb and mineral elements which facilitate in treating lipid metabolism disorders including Dyslipidemia, obesity, etc. The formulation disclosed herein may also be used in treating any condition associated with Dyslipidemia such as cardio vascular diseases, cerebro vascular diseases, stroke, pancreatitis, steatohepatitis, atherosclerosis, hyperglycemia, metabolic syndrome, etc. Further, the disclosed formulation may also be instrumental as anti-atherosclerotic and hypolipidemic agent.

The prior arts described above, indicate that some studies have been carried out into effectiveness of the various herbs individually, in the management of problems related with digestion. But still there is a need to develop herbal formulation, which can have a combined effect as a treatment option for digestion that is safe for long term use and can be used for various ailments apart from indigestion like blood pressure, asthma, cancer, inflammation, headache, ear pain and abdominal pain. In other words, there is a need to develop a synergistic formulation useful for treating digestive problems which can be administered orally as a single capsule.

### SUMMARY OF THE INVENTION

In the view of the foregoing disadvantages inherent in the known types of compositions now present in the prior art, the present invention provides an herbal supplement composition and method for improved digestion of food. As such, the general purpose of the present invention, which will be described subsequently in greater detail, is to provide a new and improved herbal composition and method for digestion of food which has all the advantages of the prior art and none of the disadvantages.

The present invention discloses a safe, natural, and environmental-friendly composition and method for digestion of food. More specifically, in one embodiment, the present invention is an herbal supplement composition for improved digestion. In another embodiment the composition of four ingredients is provided in a stabilized form.

The ingredients present in said compositions consist of asafetida, Foeniculum vulgare seeds powder, Trachyspermum ammi seeds powder and shilajit powder. Asafetida is present in the concentration between 5 mg and 5 gm. Foeniculum vulgare seeds powder is present in the concentration between 5 mg and 15 gm. Trachyspermum ammi seeds powder is present in the concentration between 5 mg and 15 gm and shilajit powder is present in the concentration between 5 mg and 300 mg.

In another embodiment of the present invention, the herbal supplement composition is provided in the form of a powder, a tablet or a capsule.

In one of the preferred embodiments of the present invention, the ingredients, asafetida, Foeniculum vulgare seeds powder, Trachyspermum ammi seeds powder and shilajit powder are mixed in a combination. Moreover, the invention develops an herbal supplement composition and method for improving the process of digestion in a subject in need. The composition comprising four ingredients are mixed together and are provided to the subject in need. In yet another embodiment, the composition is orally administered daily to the subject in need. Furthermore, the composition can be orally administered as a single dose to the subject in need. Moreover, the composition can be orally administered in multiple doses to the subject depending upon the requirement of the subject in need.

In another embodiment, the present invention is a method for improving the process of digestion in a subject in need. The method comprises oral administration of the herbal supplement composition to the subject in need. The composition comprises asafetida, Foeniculum vulgare seeds powder, Trachyspermum ammi seeds powder and shilajit powder. The concentration of asafetida in the composition ranges between 5 mg and 5 gm. The concentration of Foeniculum vulgare seeds powder in the composition ranges between 5 mg and 15 gm. Furthermore, the concentration of Trachyspermum ammi seeds in the composition ranges between 5 mg and 15 gm and the concentration of shilajit powder in the composition ranges between 5 mg and 300 mg.

In one of the preferred embodiment of invention, the present invention discloses an herbal supplement composition and method for improved digestion of food.

### DETAILED DESCRIPTION OF THE INVENTION

The embodiments herein and the various features and advantageous details thereof are explained more fully with reference to the non-limiting embodiments. Descriptions of well-known components and processing techniques are omitted so as to not unnecessarily obscure the embodiments herein.

The present invention relates to an herbal supplement composition and method for improving the process of food digestion. Moreover, the present invention provides an effective herbal composition of four herbal ingredients which are capable of enhancing the process of food digestion. The composition can be orally administered to the subject in need.

The herbal supplement composition of the preferred embodiment of this invention includes asafetida, Trachyspermum ammi seed powder, Foeniculum vulgare seed powder, and shilajit powder. None of the prior arts have yet discovered the extreme effectiveness of this herbal Supplement composition for improving the process of digestion of food in human beings. The mixture of four ingredients improves the secretion of digestive enzymes promoting the digestion of food. These ingredients improve digestion by improving the movement of digested food and fibre, making the digested food bulky, and promoting smoother passage of digested food which results in decreased chances of constipation. The herbal composition is altogether a new and novel composition till date.

Accordingly, in another embodiment, the invention is an herbal composition comprising of four different herbal ingredients mixed together to form an effective composition for improving food digestion process in human beings. The composition consists of four ingredients namely, asafetida, Foeniculum vulgare seeds powder, Trachyspermum ammi seeds powder and shilajit powder. All the four ingredients are mixed in different concentrations. The concentration of asafetida is between 5 mg and 5 gm, the concentration of Foeniculum vulgare seeds powder is between 5 mg and 15 gm, the concentration of Trachyspermum ammi seeds powder is between 5 mg and 15 gm and the concentration of shilajit powder is between 5 mg and 300 mg.

In another embodiment of the present invention, the herbal composition is incorporated within a capsule using known techniques, however the composition may be mixed together and formed into a tablet, or administered in dry powder form. In the preferred embodiment, each herbal ingredient is taken in dry powder form in a proportion which has concentration of asafetida is between 5 mg and 5 gm, the concentration of Foeniculum vulgare seeds powder is between 5 mg and 15 gm, the concentration of Trachyspermum ammi seeds powder is between 5 mg and 15 gm and the concentration of shilajit powder is between 5 mg and 300 mg. The ingredients are then preferably mixed together.

The herbal supplement composition is to be administered orally by capsule, tablet or powder form. The capsule or tablet is administered daily, weekly or monthly to the subject in need in order improve the process of digestion in the subject in need. Furthermore, the herbal composition is orally administered as a single dose to the subject in need and also the composition is orally administered in multiple doses to the subject in need as per the requirement. While the invention has been described in connection with a preferred embodiment it will be understood that it is not intended that the invention be limited to that embodiment. On the contrary, it is intended to cover all alternatives, modifications and equivalents as may be included within the spirit and scope of the invention as disclosed.

The preferred embodiment of the present invention is to develop a method for improving digestion of food in a subject comprising oral administration of the composition to the subject in need. The orally administered composition comprises of four herbal ingredients. The concentrations of the four ingredients make up the whole an effective composition for improving the food digestion process. The four herbal ingredients are namely asafetida, Foeniculum vulgare seeds powder, Trachyspermum ammi and shilajit powder. The concentration of the first ingredient, asafetida in the herbal composition is between 5 mg and 5 gm, the concentration of the second ingredient, Foeniculum vulgare seeds powder is between 5 mg and 15 gm, the concentration of the third ingredient, Trachyspermum ammi seeds powder is between 5 mg and 15 gm and lastly the concentration of the fourth ingredient, shilajit is between 5 mg and 300 mg.

As to the manner of usage and operation of the instant invention, same should be apparent from the above disclosure, and accordingly no further discussion relevant to the manner of usage and operation of the instant invention shall be provided.

Therefore, the foregoing is considered illustrative of only the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The foregoing discussion is illustrative of the invention. However, since many embodiments of the invention can be made without departing from the spirit and scope of the invention, the invention resides wholly in the claims hereinafter appended.

What is claimed is:

1. A herbal supplement composition for improved digestion of food in a subject in need having range of ingredient concentrations comprising;
Asafetida between 5 mg and 5 gm;
Foeniculum vulgare seeds powder between 5 mg and 15 gm;
Trachyspermum ammi seeds powder between 5 mg and 15 gm; and
Shilajit powder between 5 mg and 300 mg.

2. The composition of claim 1, wherein said composition is provided in the form of a powder a tablet or a capsule.

3. The composition of claim 1, wherein said ingredients are mixed in a combination.

4. The composition of claim 1, wherein said composition is orally administered daily to said subject in need.

5. The composition of claim 1, wherein said composition is orally administered weekly to said subject in need.

6. The composition of claim 1, wherein said composition is orally administered monthly to said subject in need.

7. The composition of claim 1, wherein said composition is orally administered as a single dose to said subject in need.

8. The composition of claim 1, wherein said composition is orally administered in multiple doses to said subject in need.

9. A method of improving digestion of food in a subject in need comprising orally administering a composition containing asafetida, Foeniculum vulgare seeds powder, Trachyspermum ammi seeds powder and shilajit powder.

10. The method of claim 12, wherein said composition comprises the concentration of asafetida between 5 mg and 5 gm.

11. The method of claim 12, wherein said composition comprises the concentration of Foeniculum vulgare seeds powder between 5 mg and 15 gm.

12. The method of claim 12, wherein said composition comprises the concentration of Trachyspermum ammi seeds powder between 5 mg and 15 gm.

13. The method of claim 12, wherein said composition comprises the concentration of shilajit powder between 5 mg and 300 mg.
